(19) United States
(12) Patent Application Publication
(10) Pub. No.: US 2021/0393394 A1
(43) Pub. Date: Dec. 23, 2021

(54) MULTIPLE DRIVE INTERFERENCE SCREW SYSTEM AND DEVICE

(71) Applicants: Thomas Zink, San Antonio, TX (US); Stephen O'Brien, New York, NY (US)

(72) Inventors: Thomas Zink, San Antonio, TX (US); Stephen O'Brien, New York, NY (US)

(21) Appl. No.: 17/355,357

(22) Filed: Jun. 23, 2021

Related U.S. Application Data
(60) Provisional application No. 63/042,992, filed on Jun. 23, 2020.

(51) Int. Cl. A61F 2/08; A61B 17/04; A61B 17/86; A61L 31/02

(52) U.S. Cl. CPC A61F 2/0811; A61B 17/0401; A61B 17/8615; A61B 17/8635; A61L 31/022; A61B 2017/0464; A61F 2002/0858; A61F 2002/0888; A61B 2017/0411; A61B 2017/0441; A61B 2017/0445; A61F 2002/0817

(57) ABSTRACT

A multiple drive interference screw device with a head having multiple insertion features. The screw component has a tubular body with a first end and a closed second end, and the head is positioned at the first end and configured to engage with a driver to advance the screw into the bone A penetrating tip is positioned at the second end. The head of the screw component has at least three insertion features, such as a hex shaped insertion feature around the major diameter of the head and a hexalobe shaped insertion feature within the hex shaped insertion feature, and a sprocket shaped insertion feature positioned exterior to the hex shaped insertion feature.

### CROSS-REFERENCE TO RELATED APPLICATION

The present application claims priority to, and the benefit of, U.S. Provisional Application No. 63/042,992, which was filed on Jun. 23, 2020 and is incorporated herein by reference in its entirety.

### FIELD OF THE INVENTION

The present invention relates generally to a multiple drive interference screw or fastening device for anchoring or otherwise securing a tendon or ligament implant in an opening in a bone. The screw has a screw body provided with a head at one end and a penetrating end at the opposite end, and the body has an outer threading for engagement with the bone. More specifically, the multiple drive interference screw or fastening device is designed to provide a surgeon with multiple configurations for inserting an interference screw and/or removing a stripped interference screw during a revision or repair surgery, thereby reducing the likelihood of complications from the removal of the original screw. Accordingly, the present disclosure makes specific reference thereto. Nonetheless, it is to be appreciated that aspects of the present invention are also equally applicable to other like applications, devices and methods of manufacture.

### BACKGROUND OF THE INVENTION

By way of background, interference screws or fasteners have the purpose of anchoring a transplant or implant of a tendon or ligament in or to a bone. A channel is formed in the bone in which the transplant is to he placed. The interference screw or fastener is provided to be screwed and inserted into the intermediate space between the transplant and the inner wall of the channel, so that the tendon transplant is clamped or otherwise held between the screw and the wall. The forces acting on such a tendon or ligament, for example the cruciate ligaments in a knee joint, are considerable so that the clamping force must be correspondingly large enough to guarantee the creation of a durable anchor. For this purpose, the interference screw is provided with an outer threading which penetrates into the bone material on the inner side of the channel. At the same time, the outer threading engages with the transplant to he anchored. The interference screws, which usually have a screw body provided with a head at one end for receiving a driver or insertion tool and a penetrating end at the opposite end and the body with an outer threading (or threaded shank portion), are threaded into the bone.

Currently interference screws have one feature for insertion and removal of the implant. Typically, most interference screws have an internal hexagonal shape or feature to drive the implant internally, but do not contain an external feature. Specifically, current interference screws can be shaped as a hexalobal, hex or slot, but usually do not contain more than one shape or feature. Thus, if the main feature of the interference screw strips or becomes otherwise unusable, the implant and fastener can be very difficult to remove and may cause harm to the patient.

Consequently, there is a long felt need in the art for an interference screw or fastening device that provides multiple means for insertion and/or interference screw removal. Thus, if one of the engagement features of the screw fails, a user has additional engagement features for removing the interference screw in vivo. Finally, there is a long felt need in the art for a multiple drive interference screw device that is relatively inexpensive to manufacture, and that is both safe and easy to use.

The subject matter disclosed and claimed herein, in one embodiment thereof, comprises a multiple drive interference screw or fastening device that has a screw body component with a head with multiple insertion or engagement features. The screw or fastener body component comprises a first end and a closed second end in which the head is positioned at the first end and is configured to engage with a driver or insertion tool to advance the interference screw or fastener into the bone. A penetrating tip is positioned at the second (opposite) end of the fastener body. The screw body component further comprises an outer threading which extends radially outwardly from the body. The head of the screw body component further comprises at least three insertion or securing features. Specifically, the head of the screw body component comprises an internal hexalobe shape or feature to drive the implant internally, an external hex shape or feature to drive the head of the implant externally, and an external sprocket feature with six fingers or grooves to drive the implant from the main body of the interference screw or fastener. Therefore, the interference screw device can be advanced or removed via a driver or insertion/removal tool component that mates with at least one of the engagement features in the head of the screw or fastener. Finally, if all of these engagement features fail, the surgeon can utilize the internal groove within the screw head Which allows for the insertion of a hook, grapple, crook or similarly shaped instrument to grab the implant and pull it directly out.

While this specification makes specific reference to the interference screw or fastening device of the present invention as a way in which to insert and/or remove a stripped interference screw, it will be appreciated by those of ordinary skill in the art that aspects of the present invention are also equally amenable to other like applications and other such surgical screws and/or insertion/removal issues.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some concepts in a simplified form as a prelude to the more detailed description that is presented later.

The subject matter disclosed and claimed herein, in one aspect thereof, comprises a multiple drive interference screw or fastening device having a screw body component with a head With multiple insertion or engagement features. The screw component comprises a tubular body having a first end and a closed second end. The tubular body has an interior tubular wall defining a cavity in which the thickness of the wall in a radial direction is smaller than an inner radius of the cavity. The tubular body further includes an exterior thread on an exterior tubular surface portion of the tubular wall. A head is provided at the first end and is configured to engage with a driver or other tool to advance the interference screw or fastener into the bone, and a penetrating tip at the second end with a cutting or boring element provided on the penetrating tip. The tubular body, the head and the penetrating tip are formed as a single, unitary piece.

The head of the screw or fastener component further comprises at least three insertion or engagement features or drives. In one embodiment, the head comprises an internal hexalobal shape or feature to drive the implant internally, an external hex shape or feature to drive the head of the implant externally, and an external sprocket with six fingers or grooves to drive the implant externally. Therefore, the interference screw component can be advanced, removed or adjusted via a driver tool component that mates with the internal hexalobe feature, the eternal hex feature or the external sprocket feature.

In an alternative embodiment, the present invention may comprise an interference screw component with a head comprising an internal groove that would allow for the insertion of a hook, grapple, crook or similarly shaped instrument to grab the implant and pull it directly out of the bone. The screw component would then be advanced or removed via a driver component that engages with the internal groove, wherein the driver component is a hook-shaped instrument.

In a further embodiment of the present invention, a method for inserting a surgical fastener is provided and includes the steps of providing a surgical fastener, the fastener having a body, a first end disposed at one end of the body and a second end opposite the first end of the body, with the head having at least one engagement feature. The body further has a cavity. Next, at least one of the engagement features is engaged with a tool. The tool drives the fastener into a bone such that the cavity removes a portion of the bone. Finally, the fastener secures a muscle or ligament to the bone.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a side perspective view of one potential embodiment of the multiple drive interference screw or fastener device of the present invention in accordance with the disclosed architecture, exhibiting an external hex feature, an internal hexalobe feature and an external sprocket feature;

FIG. 2 illustrates a top perspective view of one potential embodiment of the multiple drive interference screw or fastener device of the present invention in accordance with the disclosed architecture, exhibiting multiple different drive options;

FIG. 3 illustrates a perspective view of one potential embodiment of the multiple drive interference screw for fastener device of the present invention in accordance with the disclosed architecture, in which the device has an internal hexalobe feature, an external hex feature and an external sprocket feature;

FIG. 4A illustrates a side perspective view of one potential embodiment of the multiple drive interference screw device of the present invention in accordance with the disclosed architecture, in which the device has an internal groove for receipt of a hook or other shaped instrument to grab the device and pull it directly out;

FIG. 4B illustrates a side perspective view of one potential embodiment of the multiple drive interference screw device of the present invention in accordance with the disclosed architecture, illustrating the hook referenced in FIG. 4A inserted in the internal groove; and

FIG. 5 illustrates a block diagram showing an exemplary method of using one potential embodiment of the multiple drive interference screw device of the present invention in accordance with the disclosed architecture.

### DETAILED DESCRIPTION OF THE INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

Generally stated, and in one embodiment thereof, the present invention discloses a multiple drive interference screw or surgical fastening device comprising a screw or fastener component with a head having multiple insertion or securement features. The screw component includes a tubular body having a first end and a second end wherein the head is positioned at the first end and configured to engage with a driver or insertion tool to advance the screw in to the bone, and a penetrating tip is positioned at the second end. The head of the screw or fastener component further comprises at least three insertion features, such as an internal he feature, an external hex feature and an external sprocket feature. Therefore, the interference screw component can be advanced or removed via a driver or tool component that mates with the hex shaped, sprocket-shaped or hexalobe-shaped feature.

In an alternative embodiment, the present invention may comprise various configurations of the screw head. For example, the screw head can comprise a hexalobe insertion feature within a hex-shaped insertion feature, along with a sprocket feature that is exterior to the hex-shaped feature. Thus, the screw head has a hex-shaped feature around the major diameter of the head and a hexalobe-shaped feature within the hex-shaped feature and, a further sprocket shaped feature exterior to the hex-shaped feature, or the screw or the fastener head can comprise any such configuration as is known in the art based on the preferences of the user or requirements of the surgery. Further, the screw head also includes an internal groove that would allow for the insertion of a hook, grapple, crook, or similar shaped instrument to grab the implant and pull it directly out of a patient's bone.

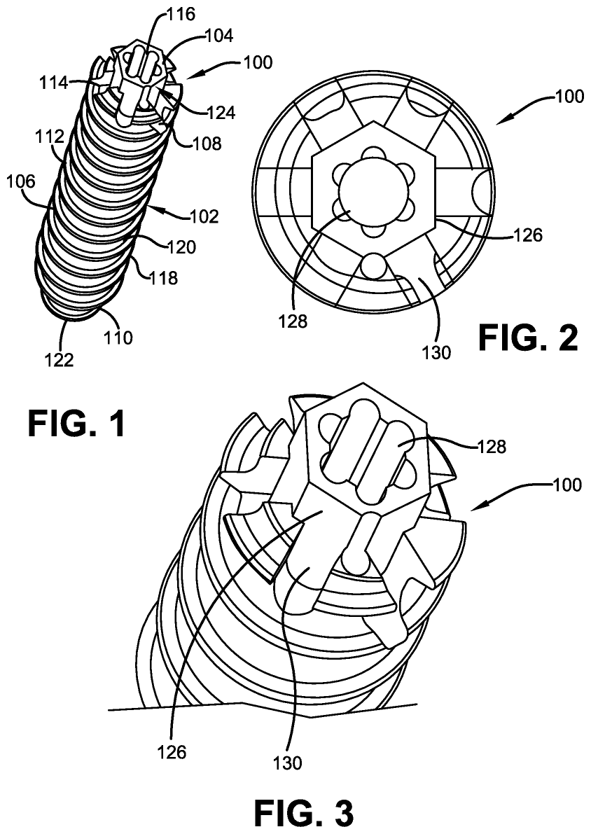

Referring initially to the drawings. FIG. 1 illustrates a side perspective view of one potential embodiment of the multiple drive interference screw or fastener device 100 of the present invention in accordance with the disclosed architecture, exhibiting an external hex feature, an internal hexalobe feature and an external sprocket feature. More specifically, the drive interference screw device 100 comprises a screw or fastener component 102 with a head 104 comprising multiple insertion features 124. Both the screw component 102 and the head 104 may be manufactured using typical machining techniques as are known. in the art and constructed as one part. Notwithstanding, the screw or fastener component 102 and head 104 are preferably manufactured from titanium, specifically Ti 6 Al 4 V-ELI, though the same can be manufactured froth any other suitable medical grade material as is known in the art. For example, the screw component 102 and head 104 could also be additively manufactured from PEEK (Polyether ether ketone) or other suitable materials.

Additionally, the multiple drive interference screw device 100 and its components can be of any suitable size, shape and configuration as is known in the art without affecting the overall concept of the invention. One of ordinary skill in the art will appreciate that the shape and size of the multiple drive interference screw or surgical fastening device 100 as shown in FIGS. 1-4 is for illustrative purposes only, and many other shapes and sizes of the multiple drive interference screw device 100 are well within the scope of the present disclosure. Although dimensions of the multiple drive interference screw device 100 (i.e., length, width, and height) are important design parameters for good performance, the multiple drive interference screw or surgical fastening device 100 may be of any shape, size or configuration that ensures optimal performance during use.

As shown in FIGS. 1-4B, the screw or fastener component 102 of the multiple drive interference screw device 100 comprises a tubular body 106 having a first end 108 and a closed second end 110. The tubular body 106 has a tubular wall 112 defining an interior cavity 114 in which the thickness of the wall 112 in a radial direction is smaller than an inner radius 116 of the cavity 114. The tubular body 106 farther includes an exterior thread 118 on an exterior tubular surface portion 120 of the tubular wall 112, extending radially outwardly from the body. The threads extend substantially the length of the body and come to terminus before the periphery of the head. The diameter of the threaded portion reduces as the threads approach the bottom end of the screw or fastener. A head 104 is disposed at the first end 108 and is configured to engage with a driver (not shown) to advance (or, when need be, withdraw) the interference screw component 102 into the bone, and a penetrating tip 122 at the second end 110. The tubular body 106, the head 104 and the penetrating tip 122 are formed as a single piece. The penetrating tip and interior cavity work together to remove a portion of the bone as the screw or fastener is inserted in to the bone.

Additionally, the head 104 of the interference screw component 102 further comprises at least three insertion features or drives 124, though more than three insertion features 124 can be utilized depending on the requirements or preferences of a user. The insertion features 124 are a series of indentations or detents located across the diameter of the head 104 for receipt of a portion of a driver or insertion device as described more fully below. The series of detents or indentations form a hexalobal pattern. The insertion features 124 are preferably any combination of hexagon, hexalobe or sprocket to ensure a secure fit with. a portion of the driver, though other geometric shapes may also be used, provided that they correspond to the shape and size of the end of the driver. Additionally, the insertion features 124 can comprise multiple shapes layered within each other, such as having a hexalobe on the inside of the head 104 and a hex around the major diameter of the head, or the opposite orientation as well.

As shown in FIGS. 1-4B, the interference screw head 104 preferably comprises a hex-shaped insertion feature 126 around the major diameter of the head 104 and a hexalobe-shaped insertion feature 128 within the hex-shaped insertion feature 126. The insertion feature 128 is provided at an end of the cavity. Further, a sprocket-shaped insertion feature 130 is positioned exterior to the hex-shaped insertion feature 126. Therefore, the interference screw component 102 can be advanced or removed via a driver component that mates with the hex-shape 126, or a mating hexalobe-shaped 128 driver component, or a mating sprocket-shaped 130 driver component.

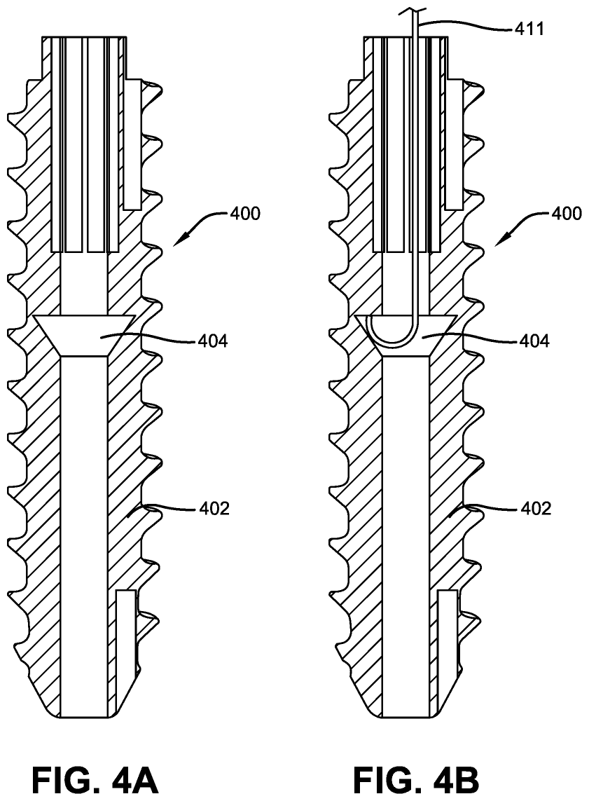

In an alternative embodiment shown in FIG. 4A, the present invention may comprise an interference screw or fastener component 400 with a body 402 having an internal groove 404 that would allow for the insertion of a hook, grapple, crook or similarly-shaped instrument 411 (see FIG. 4B) to grab the implant and pull it directly oat of the patient's bone. The internal groove 404 would be positioned around the internal diameter of the screw body 402 and would be dove-tailed in shape. To remove the hook, grapple, crook or similarly-shaped tool 411 is inserted into the cavity and engages the groove 404.

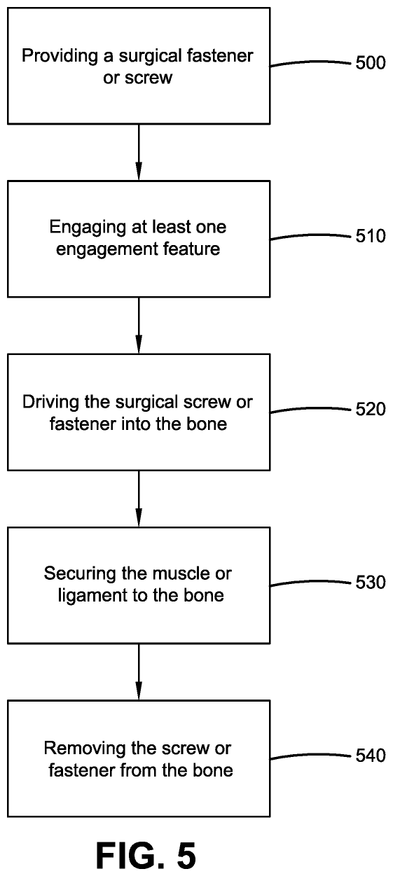

FIG. 5 illustrates a block diagram showing an exemplary method of using one potential embodiment of the multiple drive interference screw device of the present invention in accordance with the disclosed architecture. The method includes the steps of initially providing a surgical screw or fastener at step 500. Next, at least one of the driving elements is engaged by a driver at step 510, and the surgical. screw or fastener device 100 is driven into the bone at step 520. Muscle or tendon is secured to the bone at step 530 with the fastener or screw device 540, and, when required, at step 540 the screw or fastener 100 may be removed from the patient's bone.

Certain terms are used throughout the following description and claims to refer to particular features or components. As one skilled in the art will appreciate, different persons may refer to the same feature or component by different names. This document does not intend to distinguish between components or features that differ in name but not structure or function.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A surgical screw system comprising;
a screw having a body, a top end, a bottom end and an internal cavity;
a penetrating tip positioned at the bottom end, the penetrating tip in communication with the internal cavity;
the top end having a periphery and an inset area leading to the internal cavity; and
at least one insertion tool engagement feature provided on the top end, the at least one engagement feature provided on the periphery of the top end.

2. The surgical screw system as recited in claim 1, wherein the at least one tool engagement feature includes a series of detests disposed around the periphery.

3. The surgical screw system as recited in claim 2, wherein the series of detents form a hexalobal feature.

4. The surgical screw system as recited in claim 1 comprising at least three tool engagement features.

5. The surgical screw system as recited in claim 4, wherein the at least three tool engagement features include a hexalobe feature, an external hex feature, and an external sprocket feature.

6. The surgical screw system as recited in claim 5 further comprising a hook, wherein the internal cavity comprises a groove for receipt of the book as a means to remove the screw.

7. The surgical screw system as recited in claim 1, wherein the screw is manufactured from Ti 6 A14 V-ELI.

8. The surgical screw system as recited in claim 1, wherein the body of the screw comprises a threaded portion extending radially outwardly from the body.

9. The surgical screw system as recited in claim 8, wherein the threaded portion extends substantially along a length of the body and comes to terminus before the periphery of the top end of the screw.

10. The surgical screw system as recited in claim 9, wherein the threaded portion has a diameter which reduces as the threaded portion comes to the bottom end of the body.

11. A method for inserting a surgical fastener into a bone comprising the steps of:
providing the surgical fastener having a body, a first end, a second end and a head, wherein the head comprises at least one engagement feature and the body comprises an internal cavity;
engaging the at least one engagement features with a tool;
using the tool to drive the surgical fastener into the bone such that the internal cavity removes a portion of the bone; and
securing a muscle or ligament to the bone.

12. The method as recited in claim 11, wherein the internal cavity includes a groove.

13. The method as recited in claim 12 comprising a further step of removing the surgical fastener from the bone after the step of using.

14. The method as recited in claim 13, wherein the step of removing includes inserting an extraction tool into the internal cavity to connect with the groove.

15. The method as recited in claim 14, wherein the extraction tool is one of a hook, a grapple or a crook.

16. The method as recited in claim 11, wherein the step of using is performed by using one of a plurality of engagement features.

17. The method as recited in claim 16, wherein the plurality of engagement features comprises a hexalobe feature, an external hex feature and an external sprocket feature.

18. A multiple drive interference screw device comprising:
a screw comprised of a body, a first end and a second end, wherein the first and second ends are disposed at opposite ends of the body;
a cavity within the body and extending between the first end and second end;
a penetrating tip disposed at the second end of the screw; and
at least three tool engagement features disposes at the first end of the screw.

19. The multiple drive interference screw device as recited in claim 18, wherein the at least three engagement features comprise a hexalobe feature, an external hex feature and an external sprocket feature.

20. The multiple drive interference screw device as recited in claim 18 further comprising a removal tool, wherein the cavity further comprises a groove for use with the removal tool and the removal tool is one of a hook, a grapple or a crook.
